(19) United States
(12) Patent Application Publication
(10) Pub. No.: US 2015/0157856 A1
(43) Pub. Date: Jun. 11, 2015

(54) Apparatus for electroporation

(71) Applicant: Allan Westersten, Georgetown, CA (US)

(72) Inventor: Allan Westersten, Georgetown, CA (US)

(21) Appl. No.: 13/998,299

(22) Filed: Oct. 18, 2013

(51) Int. Cl. A61N 1/32; A01G 7/04

(52) U.S. Cl. CPC A61N 1/327; A01G 7/04

(57) ABSTRACT

An apparatus to make cell walls permeable to macromolecules and nanoparticles by creating micropores in cell walls without using metallic electrodes. Electrolysis and over-heating of the target material owing to uneven current distribution is eliminated. The absence of invasive electrodes makes the apparatus suitable for human applications.

### TECHNICAL FIELD OF THE INVENTION

This invention relates to apparatuses for transporting materials through cell walls, and specifically to apparatuses that do not use metallic electrodes to accomplish the above.

### BACKGROUND OF THE INVENTION

The phenomenon of electroporation of cell walls is well known. Since the 1980s, many efforts have been made to increase the transfection efficiency of poration while reducing cell death.

It has been found that there is a lower pulse energy threshold below which electroporation does not occur, and an upper threshold above which cell wall permeability becomes irreversible, resulting in cell death. There is an even higher energy threshold above which the porated cells are destroyed by overheating.

The lower threshold for an effective transmembrane potential is thought to be ca. 1.0 V, as reported by Walters et al. in U.S. Pat. No. 6,010,613 and by Weaver, in “Emerging Electromagnetic Medicine”, O'Connor et al., Editors. Other investigators report thresholds that are higher by at least an order of magnitude. The inconsistent numbers can be explained by examining the methods that have been used to arrive at the reported results. The electric field strength is usually calculated from the equation E=V/d, where V is the voltage imposed on the electrodes, and d is the distance between them. For this equation to yield accurate results for the majority of the cells between the electodes, the voltage drop between the electrodes must be uniform. That presupposes that the inter-electrode impedance is uniform. In practice, polarization at the tissue-to-electrode interfaces makes the impedance non-uniform. The impedance is highest near the electrodes, where the greatest voltage drop occurs. Mid-way between the electrodes, the voltage across the cells is much lower. Thus, when the voltage is adjusted to an appropriate level between the electrodes, it is high enough to destroy the cells near the electrodes. Conversely, when the voltage is adjusted to be appropriate near the electrode, it is below the poration threshold between the electrodes. The use of electrode voltage as a guiding parameter for developing electroporation protocols is therefore not ideal.

Westersten et al. in U.S. Pat. No. 7,664,545 disclose an apparatus that uses current, rather than the inter-electrode voltage as a basis for developing porating protocols. An increase in poration efficiency of ca. 400% was observed, compared to the conventional methods. The current pulses were delivered from a constant-current source, which made the current intensity independent of the inter-electrode impedance. Nevertheless, histological examination of the porated tissue revealed, that further improvements of the poration apparatus is desirable.

An improved poration apparatus with a high degree of poration efficiency and minimal cell damage is needed for gene therapy, for delivering chemotherapeutic agents to localized areas, for genetic engineering involving both animal and plant species and for general genetic research. It is the object of this invention to provide such an apparatus.

### Objects and Advantages

It is an object of the present invention to provide an apparatus for electroporation that yields maximal poration efficiency while minimizing cell damage.

It is another object of the invention to provide poration electrodes that are non-invasive.

It is yet another objective of the invention to provide an apparatus that optimizes the poration parameters by measuring the change in inter-electrode impedance which occurs when the poration threshold has been exceeded.

It is a further objective of the invention to eliminate metallic electrodes which cause electrolysis, resulting in cell death.

### SUMMARY OF THE INVENTION

The salient feature that distinguishes the present invention from prior art is that the ionic flux required to cause poration is generated in a circuit that does not include metallic electrodes. A non-metallic conduit containing an ionic fluid, an NaCl solution, e.g., forms the secondary winding of a transformer and the ions are accelerated to the requisite flux by adjusting the voltage imposed on the metallic primary winding of the transformer.

### BRIEF DESCRIPTION OF THE DRAWINGS

FIG. 1 illustrates the basic principle of the invention. A transformer 57 with a metallic primary winding 15 transfers energy to a non-metallic secondary winding 45. The winding 45, consisting of a non-conductive conduit containing an ionic fluid, is interfaced with the material 83 to be porated by means of conductive gel or spongiform barriers 51 that impede fluid escaping from the conduit but allow unimpeded ionic flux to impinge on the material 83 to be porated. A bolus of genetic material 77 is traversed by the ionic flux 63, which completes the secondary circuit of transformer 57.

FIG. 2 shows a cuvette 236 containing material 254 to be porated and a bolus of genetic material 292 between the non-metallic electrodes 211. The conduits 217 are joined to the secondary winding 45, shown in FIG. 1

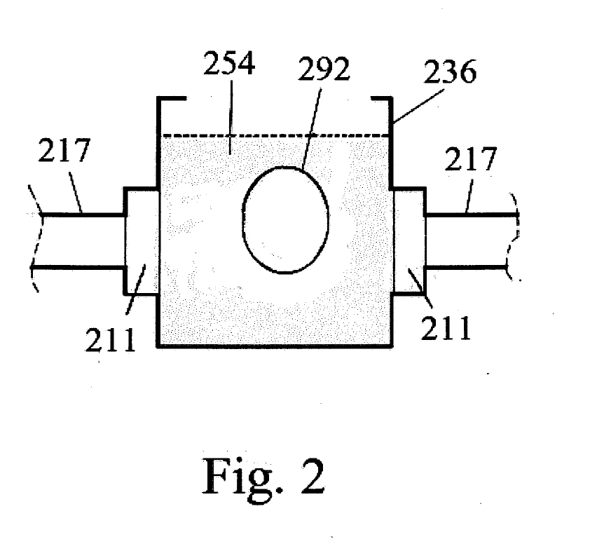

FIG. 3 shows a scaled-down version of the cuvette shown in FIG. 2, adapted to porating a single cell. The high gradient between high and low current intensities has been eliminated, making it possible to adjust the ionic flux to cause poration of a single cell without harming it.

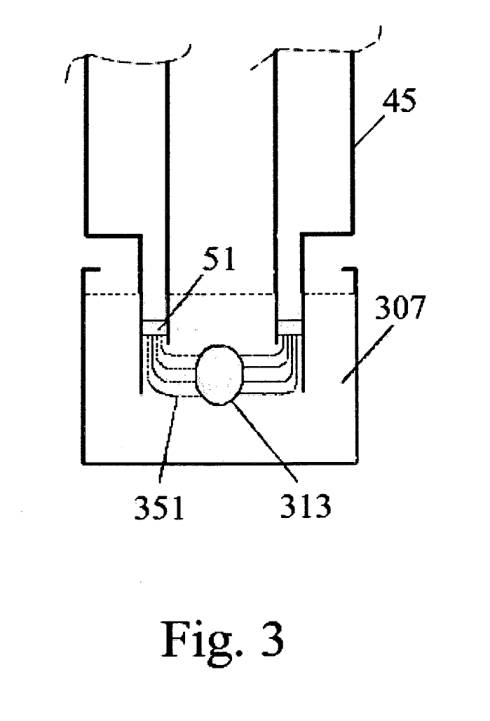

### DESCRIPTION OF THE PREFERRED EMBODIMENT

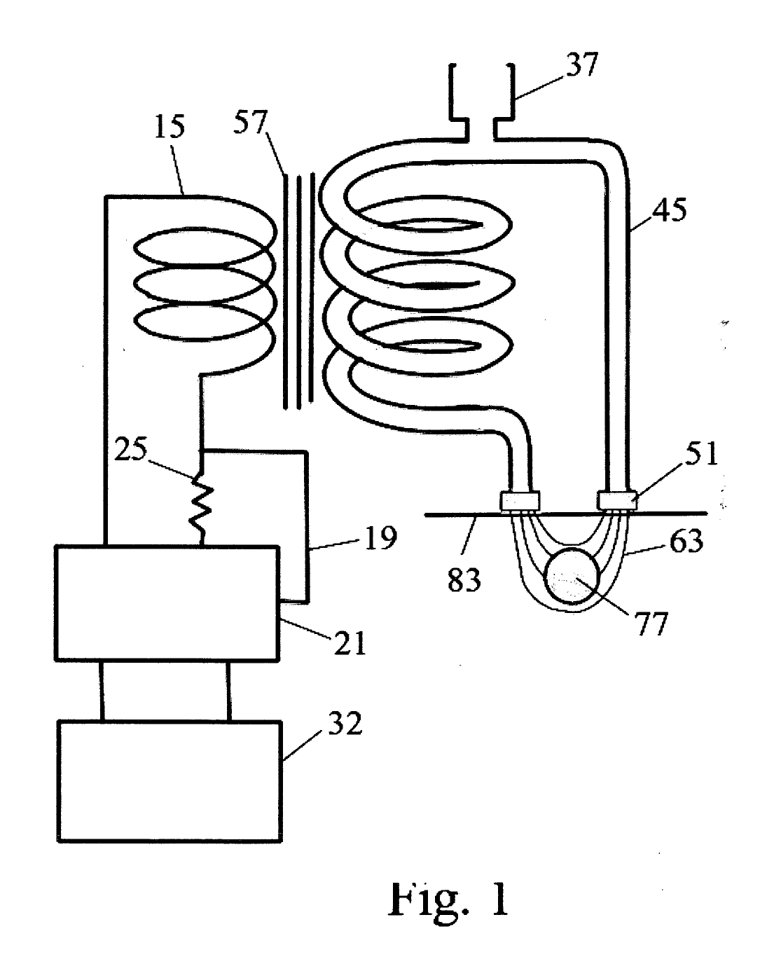

In FIG. 1, power supply 32 energizes programmable pulser 21 which causes a current flow in transformer winding 15. Current sensing resistor 25 generates a voltage proportional to the current in winding 15. When the impedance in the secondary circuit decreases owing to poration, this changes is reflected to the primary circuit and used by pulser 21 in a feedback loop, so that when the impedance in the secondary circuit is decreased, the ionic flux is prevented from increasing to destructive levels.

### RAMIFICATIONS AND SCOPE OF THE INVENTION

The above description of the preferred embodiment is not to be construed as a limitation as to the manner in which the invention may be implemented. Likewise, the application of the invention should not be limited to any one field.

The electrode system without metallic electrodes may be useful in other applications such stimulation of tissues, heart defibrillation and passive exercise equipment. I claim:

1. An apparatus for making animal and plant cells transiently permeable to macromolecules and nanoparticles by creating micropores in said cell membranes using a process termed electroporation, comprising:
a) a transformer having a metallic primary winding,
b) a non-metallic secondary winding, comprising an ionic liquid in a non-conductive conduit,
c) means to connect an ionic flux in said secondary winding to a target medium, and
d) means to energize the primary winding of said transformer with electrical waveforms of predetermined intensities and durations.
